(19) United States
(12) Patent Application Publication
(10) Pub. No.: US 2020/0216569 A1
(43) Pub. Date: Jul. 9, 2020

(54) Antigenic Proteins and Methods Therefor

(71) Applicant: NantCell, Inc., Culver City, CA (US)

(72) Inventors: Patrick Soon-Shiong, Culver City, CA (US); Shahrooz Rabizadeh, Culver City, CA (US)

(21) Appl. No.: 16/648,980

(22) PCT Filed: Sep. 28, 2018
(86) PCT No.: PCT/US2018/053379

§ 371 Date: Mar. 19, 2020

Related U.S. Application Data
(60) Provisional application No. 62/565,679, filed on Sep. 29, 2017.

(51) Int. Cl. C07K 16/44; C12N 5/0783; C07K 14/52

(52) U.S. Cl. CPC C07K 16/44; C12N 2510/00; C07K 14/52; C12N 5/0646

(57) ABSTRACT

Contemplated compositions and methods use various immunomodulatory agents to downregulate an autoimmune response and/or to upregulate immune responses against autoantigen presenting cells.

This application claims priority to our co-pending WIPO patent application with the serial number PCT/US2018/053,379, which was filed Sep. 28, 2018 and U.S. provisional patent application with the Ser. No. 62/565,679, which was filed Sep. 29, 2017.

### FIELD OF THE INVENTION

The field of the invention is compositions and methods to reduce autoimmunity against various autologous antigenic proteins, especially as it relates to Type I diabetes and Parkinson's disease.

### BACKGROUND

The following description includes information that may be useful in understanding the present invention. It is not an admission that any of the information provided herein is prior art or relevant to the presently claimed invention, or that any publication specifically or implicitly referenced is prior art.

All publications and patent applications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Type I Diabetes can be characterized as an autoimmune disease and various molecular targets for the immune system have been proposed. For example, the C-terminal portion of the zinc transporter protein ZnT8 was described as a potential immunogenic fragment as discussed in U.S. Pat. No. 9,023,984. In another approach, as described in US 2016/0361397, a composition is employed that comprises two or more overlapping fragments comprising a preproinsulin epitope, with at least one fragment being immunogenic. Here, the inventors contemplate that antigen challenge in an autoimmune setting may stimulate beneficial changes in T cell subsets (e.g., Th2 vs. Th1), in cytokine production, and/or in regulatory T cells induction, and so generate tolerance. While such and other approaches are at least conceptually attractive, a therapeutically effective regimen has not been developed using such compositions and methods. More recently, a defective ribosomal product from the human preproinsulin mRNA was described as being antigenic in the context of Type I diabetes, and various test compositions and test methods for Type I diabetes using antibodies or cytotoxic T cells against defective ribosomal products were proposed as discussed in WO2017/125586. Here, the authors also envision generation of immune tolerance against the defective ribosomal product to so treat Type I diabetes.

Therefore, even though various methods of generating immune tolerance are known in the art, there is still a need for improved compositions and methods for immune therapy to treat or ameliorate various autoimmune diseases, and especially Type 1 diabetes.

### SUMMARY OF THE INVENTION

The inventive subject matter is directed to various immune modulating compositions and methods in which immune modulation is targeted with respect to autoantigens, and especially to mRNA expression products of sequences encoding insulin and α-synuclein.

In one aspect of the inventive subject matter, the inventors contemplate a chimeric immune modulating molecule, and nucleic acids encoding same, that includes an affinity portion coupled to an immune suppressing portion.

Preferably, the affinity portion has a binding specificity against an autoantigen, and most preferably the affinity portion has a binding specificity against a translation product of an mRNA encoding insulin (e.g., ALT-ORF product starting at AUG₃₄₁ of the mRNA) or α-synuclein. With respect to the affinity portion it is contemplated that such portion may comprise an antibody or fragment thereof, a T cell receptor portion, a scFv, or a high-affinity peptide isolated by mRNA display. Therefore, the chimeric immune modulating molecules may also include an Fc portion. While not limiting to the inventive subject matter, preferred immune suppressing portions include IL-8, IL-34, TGF-β, and B7-H4.

Where a recombinant nucleic acid is contemplated, the sequence portion encoding the chimeric molecule may be under the control of an inducible, or constitutively active, or tissue specific promoter (e.g., pancreas-specific promoter). Such recombinant nucleic acids may be isolated fragments, or be at least part of a viral genome or at least part of a bacterial vector.

Consequently, the inventors also contemplate a pharmaceutical composition comprising the chimeric immune modulating molecule or a pharmaceutically acceptable recombinant virus (e.g., Ad5 with E2b gene deleted) comprising the recombinant nucleic acid presented herein, typically in combination with a pharmaceutically acceptable carrier.

In further contemplated aspects, the inventors also contemplate a chimeric immune modulating molecule, and nucleic acids encoding same, that includes an affinity portion that is coupled to an immune stimulating portion, wherein the affinity portion has a binding specificity against an autoantigen. Especially contemplated affinity portions have a binding specificity against a translation product of an mRNA encoding insulin (e.g., ALT-ORF product starting at AUG₃₄₁ of the mRNA) or α-synuclein. Similar to the molecule contemplated above, the affinity portion may comprise an antibody or fragment thereof, a T cell receptor portion, a scFv, or a high-affinity peptide isolated by mRNA display, and the chimeric molecule further comprises an IL15 portion, an IL15 receptor alpha chain portion, and an Fc portion. Thus, especially preferred molecules may be based on an ALT803 scaffold with an affinity portion as described above. Thus, the inventors also contemplate a pharmaceutical composition comprising the chimeric immune modulating molecule presented herein, typically in combination with a pharmaceutically acceptable carrier.

In still further contemplated aspects, the inventors contemplate a genetically engineered NK cell that includes a recombinant nucleic acid encoding at least a portion of a T cell receptor having specificity against an autoantigen. Preferably, the NK cell is a NK92 derivative, and/or the portion of the T cell receptor comprises a TCR-α, a TCR-β chain, and a CD3ζ chain. As noted above it is generally preferred that the autoantigen is a translation product of an mRNA encoding insulin or α-synuclein. Consequently, a pharmaceutical composition is contemplated that comprises the genetically engineered NK cells as presented herein. Likewise, in yet further contemplated aspects, the inventors contemplate also uses and methods of chimeric immune modulating molecules and modified NK cells as described herein to treat an autoimmune disease, and especially Type 1 diabetes or Parkinson's disease.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is an exemplary mRNA sequence encoding insulin in which translation products are depicted below the mRNA sequence.

FIG. 2 is a schematic illustration of the sequence of FIG. 1 showing the potential start codons for the preproinsulin ORF and the ALT-ORF, along with a likelihood score of usage for the respective start codons.

FIG. 3 is a schematic illustration of a chimeric construct that includes affinity portions against autoimmune epitopes and an immunostimulatory portion.

### DETAILED DESCRIPTION

The inventors have discovered that various autoimmune diseases, and particularly Type I diabetes and Parkinson's disease can be treated by using neoepitopes/antigens for certain proteins that are associated with the autoimmune disease, wherein the neoepitopes or antigens are used in the context of one or more immune suppressive factors and/or cell-based constructs to attenuate an immune response and/or eradicate neoepitope or antigen presenting cells.

In particularly preferred aspects, severity of the autoimmune reaction can be reduced by a chimeric protein that has an affinity portion that binds to a protein that is associated with the autoimmune disease (e.g., the neoepitope or antigen), and that further has an immune modulatory portion that has a suppressive effect. Such chimeric protein is considered to reduce autoimmune reactive cells and to promote tolerance to the protein. To eradicate or reduce the number of autoimmune reactive antigen-presenting cells, genetically modified NK cells can be employed that have a chimeric T cell receptor (e.g., obtained from reactive T cells of a patient) that binds the MHC-bound protein associated with the autoimmune disease, which in turn will trigger NK-cell killing via release of granzyme and perforin. Alternatively, or additionally, a T×M hybrid construct can be generated that is based on ALT803 (i.e., IL-15 mutant (IL-15N72D) protein bound to an IL-15 receptor α/IgG1 Fc fusion protein) and that has an affinity portion that binds to a protein that is associated with the autoimmune disease. In that manner, NK based cell killing with specificity towards autoimmune reactive antigen-presenting cells is stimulated, which is believed to reduce or even eliminate autoimmunity.

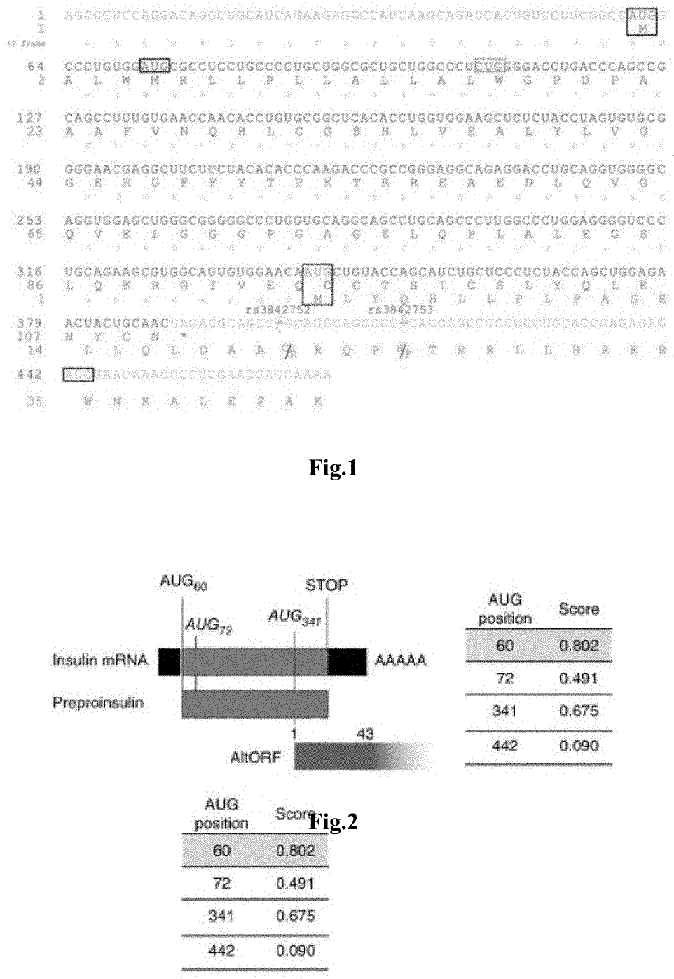

In one example of the inventive subject matter, the inventors noted that an insulin mRNA has multiple possible start codons from which genetic information can be translated into protein as is depicted in FIG. 1. While the proper start codon at position 60 will result in the formation of preproinsulin, three additional start codons are available at positions 72, 341, and 442 as is also schematically depicted in FIG. 2. Assuming that an incorrect start codon is used in a beta cell, the inventors now postulate that an insulin-based neoepitope is formed (which may be the result from a frame shift or from in-frame protein misfolding) that then becomes a trigger to an immune response against the beta cells in the pancreas. More specifically, FIG. 1 shows the full-length insulin mRNA with the bona fide PPI ORF in black uppercase letters, 5′ and 3′ UTRs in gray uppercase letters, and the poly(A) signal sequence in bold gray letters. The preproinsulin (PPI) amino acid sequence is shown in dark font (SEQ ID NO:1), the amino acid sequences with SNP variants of the +2 reading frame (SEQ ID NO:2 and SEQ ID NO:3) are shown in small, light gray font, and the amino acid sequence of the alternative open reading frame (ALT-ORF) is shown below the mRNA sequence in bold grey. All AUG codons within the mRNA are framed with a black box, and those used as translation initiation site are indicated with lighter grey corresponding to the resulting amino acid sequence. The * indicate the stop codons in the given amino acid sequence. The putative non-AUG (CUG) start site is framed with a light grey dashed line. The 3′-UTR SNPs are annotated and both polymorphisms are depicted, as are the potentially affected amino acids in the nonconventional polypeptide. As noted above, FIG. 2 depicts a schematic representation of full-length human insulin mRNA. The 5′ and 3′ UTR are depicted in black and the insulin-encoding ORF starting at AUG in dark grey. Alternative translation initiation sites are shown in italic and the poly(A) tail is indicated in bold. The ALT-ORF encoding the out-of-frame polypeptide is shown in grey, and the first amino acid (1) and the last amino acid preceding the poly(A) tail (43) are depicted. On the right side in FIG. 2, translation initiation scores are shown for every AUG codon within the insulin mRNA sequence as predicted by the NetStart 1.0 prediction server. Prediction scores greater than 0.5 are considered probable translation start codons. Further details and considerations suitable for use herein are described elsewhere (Nature Medicine 23, 501-507 (2017)). Thus, all polypeptide products originating from alternative start codon usage are considered insulin-based neoepitopes/antigens.

In view of the above, and to reduce autoimmune attack and to generate tolerance, the inventors now contemplate that a chimeric protein construct can be prepared that comprises a first portion that binds to the insulin-based neoepitope or ALT-ORF (which may also be a misfolded PPI), or may comprise a neoepitope that is based on the ALT-ORF (which may be membrane bound and/or bound on a MHC complex) as a first portion, and that has a second portion that will provide an immune suppressive effect and/or will contribute to generation of immune tolerance.

Most typically, the binding portion may either be specific to the particular autoantigen or neoepitope per se, or may be specific to the particular autoantigen/neoepitope when the particular autoantigen or neoepitope is bound to an MHC complex on an antigen presenting cell. Most typically, where the binding portion is specific to the particular autoantigen or neoepitope per se, the binding portion may be a scFv that has a known and defined affinity to the autoantigen/neoepitope. Such scFvs may be based on the V_{H} and V_{L} portions of in vivo or in vitro generated antibodies, or based on antibodies against the autoantigen/neoepitope from a patient with the autoimmune disease. Alternatively, such scFv portions may also be derived from screening a high-diversity RNA display library using the autoantigen/neoepitope as bait. In other examples, where the binding portion is specific to the particular autoantigen or neoepitope bound to an MHC complex, suitable binding portions will especially include recombinant T cell receptor alpha and beta chains (or antigen binding portions thereof). As will be readily appreciated, such T cell receptors can be isolated from T cells of a patient with the autoimmune disease following established protocols. Where the autoantigen is synuclein or a splice variant thereof, suitable sequences for alpha synuclein are found in UniProtKB under the accession number P37840, with various mRNA sequences encoding alpha synuclein found, for example, at EMBL accession numbers L08850, L36674, L36675, and D31839.

However, in further alternative aspects, it should be noted that the binding portion may also comprise an entity other than a scFv or TCR, such as a peptide or protein that binds with a high affinity (e.g., K_{D}<10⁷M) to the autoantigen/neoepitope, or an aptamer or other synthetic binder. Additionally, it should be appreciated that suitable neoepitopes may also be identified using omics analysis. Moreover, it is further preferred that autoantigen/neoepitope identified herein may be further qualified via computational analysis of binding to a patient's MHC type (e.g., using netMHC). Therefore, it should be appreciated that binding portions may be identified or prepared from various synthetic sources, and especially high-diversity libraries (e.g., RNA/phage display libraries), or by isolation of autoantigen/neoepitope reactive T cells and subsequent isolation of the T cell receptor as further described in more detail below.

Especially preferred portions that provide the immune suppressive effect and/or immune tolerance include IL-8, TGF-β, IL-27, IL-35, IL-37, or B7H4 (or portions thereof), which may be coupled to the binding portion by way of a peptide bond to form a chimeric protein. For example, suitable sequences for IL-8 can be found at UniProtKB database entry P10145, suitable sequences for TGF-β can be found at UniProtKB database entry P01137, suitable sequences for IL-27 can be found at UniProtKB database entry Q8NEV9/Q14213, suitable sequences for IL-37 can be found at UniProtKB database entry Q9NZH6, and suitable sequences for B7-H4 can be found at UniProtKB database entry Q7Z7D3. However, various other immune suppressive non-protein portions also contemplated, and especially contemplated compounds include tetracycline-type antibiotics, glucocorticoid-type drugs, tacrolimus, cyclosporine, etc. Depending on the particular molecule, the manner of covalent coupling may vary, and the PHOSITA will be well apprised of appropriate coupling agents and methods. While numerous manners of coupling are deemed suitable, particularly preferred manners include in-frame expression of a nucleic acid construct that encodes a single polypeptide chain for the scFv, an optional intervening linker sequence, and the portion that provides the immune suppressive effect.

Most typically, therefore, the binding portion will be covalently bound to the second portion that provides the immune suppressive effect and/or immune tolerance. For example, where the binding portion is a scFv and the second portion is a protein (e.g., IL-8 of TGF-β), the covalent bond may be a peptide bond in the backbone of a chimeric protein. Construction of chimeric protein will use standard methods of cloning and protein production, and may be performed in bacterial (e.g., E. coli B21 ClearColi), yeast (e.g., Pichia pasteuris, Saccharomyces cerevisiae, etc.), or eukaryotic (e.g., SF9 cell culture, CHO cells culture) production systems. Consequently, it should be recognized that recombinant nucleic acids encoding the chimeric proteins are also contemplated, and recombinant nucleic acid constructs may be linear or circular extrachromosomal nucleic acids, or recombinant nucleic acids that are integrated into a host cell genome. The sequence portion encoding the chimeric immune modulating molecule is typically under the control of a constitutively active promoter in a production environment, or under the control of a tissue specific promoter in a viral delivery environment. In another example, for viral delivery of a type 1 diabetes chimeric construct, the promoter may be a pancreas-specific promoter such as an INS (insulin) promoter, an IRS2 (Insulin receptor substrate 2) promoter, a Pdx1 (pancreatic and duodenal homeobox 1) promoter, a Alx3 (Aristaless-like homeobox 3) promoter, or a Ppy (pancreatic polypeptide) promoter.

Alternatively, it is also contemplated that a chimeric protein construct can be prepared that comprises one portion that is based on or includes the ALT-ORF of insulin, misfolded protein, or other neoepitope, and that further comprises a second portion that will provide an immune suppressive effect and/or will contribute to generation of immune tolerance. For example, such chimeric protein may include the ALT-ORF of insulin (as shown in FIGS. 1 and 2) fused to TGF-beta or IL-10 (or other portion that provides the immune suppressive effect and/or immune tolerance as described above). Contemplated chimeric products will typically, but not necessarily have a linker disposed between the first and second portions, which may be flexible, rigid, and in some cases even cleavable (see e.g., Adv Drug Deliv Rev. 2013 October; 65(10):1357-69). For example, suitable linker sequences between the first and second portions include (G₄S)ₙ-linkers with n typically between 1 and 10.

Administration of contemplated chimeric protein constructs is most typically by injection either systemically (e.g., via i. v. injection), or localized, typically into the affected tissue. The dosage and schedule can be determined using dose escalation, or generally follow physiological concentrations for the compound that effects immune suppression or tolerance. Alternatively, contemplated chimeric protein constructs may also be part of a gene therapy in which a virus containing a recombinant nucleic acid is delivered to a patient, and in which the recombinant nucleic acid is then expressed in a host cell of the patient, preferably in a tissue specific manner (e.g., using a promoter that is tissue specific to the diseased tissue). Here, administration will typically follow protocols for viral gene therapy where at least 10⁶, or at least 10⁸, or at least 10¹⁰ viral particles are transfused in a single administration.

In another aspect of the inventive subject matter, the inventors also contemplate that a chimeric antigen receptor protein can be constructed that binds the autoantigen/neoepitope and that is expressed on a cytotoxic cell, and most preferably on an NK cell. Such genetically modified cells are considered to reduce or even entirely eliminate (antigen presenting) cells that display the autoantigen/neoepitope, which in turn will reduce an autoimmune response. Most typically, the cytotoxic cell or NK cell is transfected with a recombinant nucleic acid encoding the chimeric T cell receptor, typically following protocols well known in the art. Therefore, suitable recombinant nucleic acids will include mRNA, linear dsDNA, and viral expression vectors.

For example, preferred chimeric antigen receptors will include an scFv portion or small peptide with high affinity to the autoantigen/neoepitope as an ectodomain, which is most typically coupled to transmembrane domain, that is in turn coupled to a signaling endodomain that includes a plurality of ITAM motifs. For example, suitable chimeric antigen receptors will comprise a scFv (that binds the autoantigen/neoepitope) antibody fragment, coupled to a flexible hinge region, and a CD3ζ chain. Of course, it should be appreciated that the scFv portion may also be coupled to multiple signaling domains, such as CD3ζ-CD28-41BB or CD3ζ-CD28-OX40, to increase signaling. Three are numerous method of generating and expressing chimeric antigen receptors known in the art, and all of such compositions and methods are deemed suitable for use herein (see e.g., Mol Ther. 2017 Aug. 2; 25(8):1769-1781; or J Cell Mol Med. 2016 July; 20(7): 1287-1294; or Sci Rep. 2015; 5: 11483). With respect to the neoepitope/antigen binding domain of the chimeric antigen receptor, the same considerations as noted above apply. Moreover, it should be appreciated that the chimeric antigen receptors will generally be expressed in cytotoxic cells, and especially NK cells to so deliver a target specific cytotoxic response to all cells that display the neoepitope/antigen.

In still further contemplated aspects, and particularly where the neoepitope/antigen is bound to an WIC complex on an antigen presenting cell, recombinant cytotoxic cells are contemplated that express a T cell receptor that binds to the antigen MHC/complex. Most typically, the recombinant T cell receptor can be generated from a T cell receptor of a T cell that is reactive against the autoantigen or neoepitope. Most typically, such T cells can be isolated following known protocols (e.g., Curr Opin Endocrinol Diabetes Obes. 2017 April; 24(2): 98-102; or Diabetes. 2015 January; 64(1):172-82; WO 2017/125586 and US national phase document thereof; or PLoS ONE 2011, Vol. 6(11), e27930). For example, where the cytotoxic cell is an NK cell, it is contemplated that the alpha and beta chain of the T cell receptor can be cloned and expressed from a single nucleic acid (e.g., mRNA) and that the CD3 gamma and CD3 delta subunit may be expressed from another single nucleic acid (e.g., mRNA) to so reconstruct a functional T cell receptor in the NK cell. Alternatively, all four subunits may also be co-expressed from a single mRNA (typically separated by T2A, P2A, and/or F2A sequences). In this context, it should be noted that the NK cell will typically provide endogenous CD3 zeta and CD3 epsilon domains.

Therefore, recombinant therapeutic cytotoxic cells are contemplated, and particularly recombinant NK cells, which may be allogenic NK cells or NK cells from the patient. However, it is typically preferred that the NK cells are NK92 cells or derivatives thereof. For example, particularly preferred NK cells include NK cells that are genetically modified to have a reduced or abolished expression of at least one killer cell immunoglobulin-like receptor (KIR), which will render such cells constitutively activated via lack of or reduced inhibition. Such cells may also be commercially obtained from NantKwest (see URL www.nantkwest.com) as aNK cells. Further suitable NK cells include genetically engineered NK cells that express a high-affinity Fcγ receptor (e.g., CD16, V₁₅₈), which are commercially available from NantKwest as haNK cells (high-affinity natural killer cells).

Recombinant NK cells are preferably administered in a transfusion of between about 10⁶-10⁷, or between about 10⁷-10⁸, or between about 10⁸-10⁹, or between about 10⁹-10¹⁹ (or even more) cells per transfusion. Transfusion may be done alone, or in combination with or subsequent to administration of the chimeric protein construct described above that has a portion that binds to an autoantigen/neoepitope and that has a second portion that provides an immune suppressive effect and/or will contribute to generation of immune tolerance.

In yet another aspect of the inventive subject matter, the inventors also contemplate a chimeric protein construct that has one portion that specifically binds with high affinity to an autoantigen/neoepitope (e.g., the insulin-based ALT-ORF neoepitope which may be membrane bound and/or bound on a MHC complex) and that has a second portion that provides an immune stimulatory effect to cytotoxic cells, and especially T cells and NK cells. As already noted above, the binding portion is preferably an scFv, but may be any peptide or protein that binds with high affinity to the autoantigen/neoepitope. For example, the binding portion may be derived from an isolated antibody or from a molecule isolated from an RNA or phage display method. Therefore, and most typically, the chimeric protein construct will comprise a single peptide backbone in which an immune stimulatory protein is fused in frame to the binding portion.

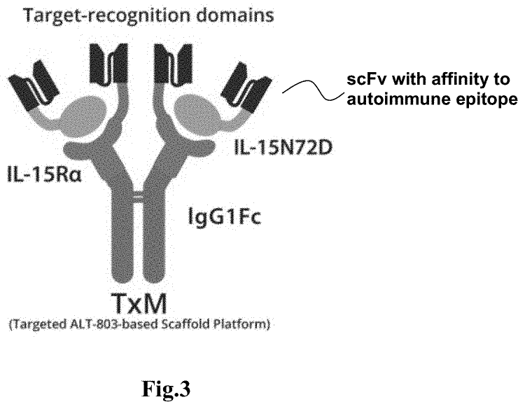

The immune stimulatory portion is preferably an immune stimulatory cytokine that activates cytotoxic cells, and especially NK cells. Therefore, preferred immune stimulatory portions will comprise at least a portion of IL-2 or IL-15, or may comprise an ALT803-type superkine that is based on an IL-15:L-15 receptor alpha superagonist complex (as described in Cytokine. 2011 December; 56(3): 804-810). In addition, such superagonist complex is modified by addition of scFv portions to at least one of the IL-15 and the IL-15 receptor alpha chain (e.g., as described in US 2018/0200366). Where the immune stimulatory portion comprises ALT803, the configuration is most preferably as a T×M as schematically shown on FIG. 3. Here, the chimeric molecule includes an Fc portion that increases serum half-life of the chimeric molecule and that provides a binding site for (high affinity) NK cells via CD16. The IL15 receptor/IL15 superagonist portion provides for a stimulatory signal for the cytotoxic cells that is bound to the autoantigen/neoepitope via the binding portion. Administration of the chimeric protein construct is typically by injection either systemically via i. v. injection, or localized, typically via intratumoral injection. With respect to dosage and schedule it is contemplated that these parameters will typically follow conventional administration schedules for ALT803.

Therefore, it should be appreciated that contemplated compositions and methods will not only allow for immune suppression/generation of immune tolerance in the specific context of the autoantigen/neoepitope, but also enable reduction or even elimination of APCs that would otherwise perpetuate an immune response against the autoantigen/neoepitope. Alternatively or additionally, in still another aspect of the inventive subject matter, a recombinant virus can be generated for gene therapy that produces, upon transcription, an antisense or siRNA that blocks translation of the autoantigen/neoepitope, and especially of the ALT-ORF (e.g., using methods as described in US 2014/0296321).

Consequently, the inventors contemplate methods of treating autoimmune diseases, and especially Type I diabetes, by administering one or more treatment compositions that include a chimeric construct that binds with one portion to the insulin-based neoepitope and that has a second, immune suppressive portion (e.g., IL-8, TGF-beta) to so generate immune tolerance. Once tolerance is established (or alternatively), a T cell receptor is cloned from T cells that are reactive to the insulin-based neoepitope. The cloned receptor is then expressed as a functional recombinant T cell receptor in NK cells that will then be transfused back to the patient to kill all cells that present the insulin-based neoepitope (typically via MHC I or II presentation).

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their end points, and open-ended ranges should be interpreted to include commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Furthermore, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used herein, the term “treat” , “treating” or “treatment” of any disease or disorder refers, in one embodiment, to the administration of one or more compounds or compositions for the purpose of ameliorating the disease or disorder (e.g., slowing or arresting or reducing the development of the disease or at least one of the clinical symptoms thereof) . In another embodiment “treat”, “treating”, or “treatment” refers to the administration of one or more compounds or compositions for the purpose of alleviating or ameliorating at least one physical parameter including those which may not be discernible by the patient. In yet another embodiment, “treat”, “treating”, or “treatment” refers to the administration of one or more compounds or compositions for the purpose of modulating the disease or disorder, either symptomatically, (e.g., stabilization of a discernible symptom), physiologically, (e.g., breaking the escape phase of cancer immunoediting, induction of an elimination phase of cancer immunoediting, reinstatement of equilibrium phase of cancer immunoediting), or both. In yet another embodiment, “treat”, “treating”, or “treatment” refers to the administration of one or more compounds or compositions for the purpose of preventing or delaying the onset or development or progression of the disease or disorder. The terms “treat”, “treating”, and “treatment” may result, for example in the case of cancer in the stabilization of the disease, partial, or complete response. However, and especially where the cancer is treatment resistant, the terms “treat”, “treating”, and “treatment” do not imply a cure or even partial cure. As also used herein, the term “patient” refers to a human (including adults and children) or other mammal that is diagnosed or suspected to have a disease, and especially cancer.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only 7one element from the group, not A plus N, or B plus N, etc.

1. A chimeric immune modulating molecule, comprising:
an affinity portion coupled to an immune suppressing portion;
wherein the affinity portion has a binding specificity against an autoantigen; and
wherein the immune suppressing portion is selected from the group consisting of IL-8, IL-34, TGF-β, and B7-H4.

2. The chimeric immune modulating molecule of claim 2, further comprising a peptide linker between the affinity portion and the immune suppressing portion.

3. The chimeric immune modulating molecule of claim 1, wherein the affinity portion has a binding specificity against a translation product of an mRNA encoding insulin or α-synuclein.

4. The chimeric immune modulating molecule of claim 3, wherein the translation product of the mRNA encoding insulin is an ALT-ORF product starting at AUG₃₄₁ of the mRNA.

5. The chimeric immune modulating molecule of claim 1, wherein the affinity portion comprises an antibody or fragment thereof, a T cell receptor portion, a scFV, or a high-affinity peptide isolated by mRNA display.

6. The chimeric immune modulating molecule of claim 1, wherein the affinity portion is coupled to the immune suppressing portion via a moiety that includes an Fc portion.

7-13. (canceled)

14. A chimeric immune modulating molecule, comprising:
an affinity portion coupled to an immune stimulating portion, wherein the affinity portion has a binding specificity against an autoantigen.)

15. The chimeric immune modulating molecule of claim 14, wherein the affinity portion has a binding specificity against a translation product of an mRNA encoding insulin or α-synuclein.

16. The chimeric immune modulating molecule of claim 15, wherein the translation product of the mRNA encoding insulin is an ALT-ORF product starting at AUG₃₄₁ of the mRNA.

17. The chimeric immune modulating molecule of claim 14, wherein the affinity portion comprises an antibody or fragment thereof, a T cell receptor portion, a scFV, or a high-affinity peptide isolated by mRNA display.

18. The chimeric immune modulating molecule of claim 14, wherein the immune stimulating portion comprises an IL15 portion, an IL15 receptor alpha chain portion, and an Fc portion.

19-22. (canceled)

23. A genetically engineered NK cell comprising a recombinant nucleic acid that encodes at least a portion of a T cell receptor having specificity against an autoantigen bound to an MHC complex.

24. The genetically engineered NK cell of claim 23, wherein the NK cells is a NK92 derivative.

25. The genetically engineered NK cell of claim 23, wherein the portion of the T cell receptor comprises a TCR-α, a TCR-β chain, and optionally at least one of a CD3 gamma and CD3 delta chain.

26. The genetically engineered NK cell of claim 23, wherein the autoantigen is a translation product of an mRNA encoding insulin or α-synuclein.

27. The genetically engineered NK cell of claim 26, wherein the translation product of the mRNA encoding insulin is an ALT-ORF product starting at AUG₃₄₁ of the mRNA.

28. A pharmaceutical composition comprising the genetically engineered NK cell of claim 26, in combination with a pharmaceutically acceptable carrier.

29-35. (canceled)
